(19) United States
(12) Patent Application Publication
(10) Pub. No.: US 2025/0100032 A1
(43) Pub. Date: Mar. 27, 2025

(54) METHOD FOR DIGESTING BIODEGRADABLE PACKAGING WASTE AND AN APPARATUS THEREOF

(71) Applicant: TRIA PTE. LTD., Singapore (SG)

(72) Inventors: Pei Kang NG, Singapore (SG); Sui Ming Raymond HON, Singapore (SG)

(21) Appl. No.: 18/564,730

(22) PCT Filed: Jun. 13, 2022
(86) PCT No.: PCT/SG2022/050405

§ 371 Date: Nov. 28, 2023

(51) Int. Cl. B09B 3/60; C12M 1/00; C12M 1/06; C12M 1/26; C12M 1/34

(52) U.S. Cl. CPC B09B 3/60; C12M 23/34; C12M 23/58; C12M 27/06; C12M 33/04; C12M 41/12; C12M 41/26; C12M 41/34

(57) ABSTRACT

The present invention relates to a method for digesting biodegradable packaging waste and an apparatus thereof. Specifically, the present invention relates to a method of treating biodegradable packaging waste prior to subjecting the biodegradable packaging waste to a biological valorisation process and an apparatus thereof. The apparatus includes a tank for receiving the biodegradable packaging waste therein, the tank comprises a top portion and a bottom portion below the top portion, an air supply module adapted to supply air into the tank, a microbe injector adapted to inject mesophilic microbe into the tank, such that the tank is adapted to receive the biodegradable packaging waste, air and mesophilic microbe therein, wherein the biodegradable packaging waste is subjected to mesophilic aerobic digestion prior to conveying the biodegradable packaging waste from the tank to a biological valorisation system to subject the biodegradable packaging waste to the biological valorisation process.

### TECHNICAL FIELD

The present invention relates to a method for digesting biodegradable packaging waste and an apparatus thereof. Specifically, the present invention relates to a method of treating biodegradable packaging waste prior to subjecting it to biological valorisation means and an apparatus thereof.

### BACKGROUND

The conveniences offered by single-use packaging for the food service sector, e.g. quick service, casual dining and fast casual restaurants, have been prevalent over the years and the use of such packaging has been increasing with the rising cost of labour and the increase rate of table turnovers. This has consequently led to the increase in use of single-use packaging for the food service sector.

When the single-use packaging is disposed, the waste, which may be made up of pulp, fibre, plastics and metal materials, therefrom is often mixed with post-consumption food waste, i.e. mixed waste. The mixed waste, i.e. packaging waste and food waste, are commonly disposed through incineration or landfill. Efforts to recycle has been difficult due to the need to segregate the waste into each material types, along with the hassle and resources involved.

With the use of fully biodegradable materials for packaging, as opposed to partial or non-biodegradable materials, both packaging waste and food waste can be put through biological valorisation processes such as enzymatic hydrolysis, fermentation, anaerobic digestion, or industrial composting to avoid the need to segregate the mixed waste.

While the waste from biodegradable packaging can be biodegraded through biological valorisation processes, they often require a long amount of time to do so. Hence, it may not be cost effective and practical compared to current industry practices. For example, some biodegradable packaging can take up to 6 months to biodegrade in industrial composting sites, which typically process food waste within 2 months. With industrial anaerobic digestion, these biodegradable packaging waste may take more than 120 days to break down, as opposed to 20-30 days with food waste. This makes it impractical to biodegrade biodegradable packaging together with food waste. Therefore, the likelihood of disposing such mixed waste through incineration or landfill is higher than biodegrading it through biological valorisation means.

Therefore, there should be a way of reducing the amount of time to break down the biodegradable packaging waste to match the time taken to break down organic waste so that it is more practical and attractive to biodegrade the biodegradable packaging waste together with food waste without segregation.

### SUMMARY

According to various embodiments, a method for treating biodegradable packaging waste prior to subjecting the biodegradable packaging waste to biological valorisation process is provided. The method includes subjecting the biodegradable packaging waste to mesophilic aerobic digestion prior to subjecting the biodegradable packaging waste to a biological valorisation process.

According to various embodiments, the mesophilic aerobic digestion of the biodegradable packaging waste may be maintained at a temperature in the range of about 20° C. to about 45° C.

According to various embodiments, the mesophilic aerobic digestion of the biodegradable packaging waste may be maintained at a pH value in the range of about 4 to about 7.

According to various embodiments, the mesophilic aerobic digestion of the biodegradable packaging waste may be maintained at a dissolve oxygen level in the range of about 2 mg/L to about 3 mg/L.

According to various embodiments, the method may further include receiving the biodegradable packaging waste in a tank comprising a top portion and a bottom portion below the top portion, supplying air into the tank, injecting mesophilic microbe into the tank and conveying the biodegradable packaging waste from the tank to a biological valorisation system.

According to various embodiments, the method may further include aerating the biodegradable packaging waste and propelling the biodegradable packaging waste from the top portion toward the bottom portion of the tank.

According to various embodiments, the method may further include circulating the biodegradable packaging waste from the bottom portion to the top portion of the tank.

According to various embodiments, an apparatus for treating biodegradable packaging waste prior to subjecting the biodegradable packaging waste to a biological valorisation process is provided. The apparatus includes a tank for receiving the biodegradable packaging waste therein, the tank comprises a top portion and a bottom portion below the top portion, an air supply module adapted to supply air into the tank, a microbe injector adapted to inject mesophilic microbe into the tank, such that the tank is adapted to receive the biodegradable packaging waste, air and mesophilic microbe therein, wherein the biodegradable packaging waste is subjected to mesophilic aerobic digestion prior to conveying the biodegradable packaging waste from the tank to a biological valorisation system to subject the biodegradable packaging waste to a biological valorisation process.

According to various embodiments, the apparatus may further include a temperature module configured to maintain the mesophilic aerobic digestion of the biodegradable packaging waste at a temperature in a range of about 20° C. to about 45° C.

According to various embodiments, the apparatus may further include a pH module configured to maintain the mesophilic aerobic digestion of the biodegradable packaging waste at a pH value in the range of about 4 to about 7.

According to various embodiments, the air supply module may be configured to supply air into the tank to maintain the mesophilic aerobic digestion of the biodegradable packaging waste at a dissolved oxygen level in the range of about 2 mg/L to about 3 mg/L.

According to various embodiments, the apparatus may further include a surface aerator disposed in the tank, wherein the surface aerator is adapted to aerate the biodegradable packaging waste and propel the biodegradable packaging waste at the top part of the waste toward the bottom part of the waste.

According to various embodiments, the apparatus may further include a circulating mechanism in fluid communication with the tank and adapted to circulate the biodegradable packaging waste from the bottom part of the waste to the top part of the waste.

According to various embodiments, the tank may be thermally insulated.

According to various embodiments, a method for digesting biodegradable packaging waste is provided. The method includes subjecting the biodegradable packaging waste to mesophilic aerobic digestion, and subsequently subjecting the biodegradable packaging waste to a biological valorisation process.

### BRIEF DESCRIPTION OF DRAWINGS

FIG. 1 shows an exemplary method for treating biodegradable packaging waste prior to subjecting the biodegradable packaging waste to a biological valorisation process.

FIG. 2 shows an exemplary embodiment of an apparatus for treating biodegradable packaging waste prior to subjecting the biodegradable packaging waste to a biological valorisation process.

FIG. 3 shows another exemplary embodiment of the apparatus.

FIG. 4 shows an exemplary embodiment of the tank.

### DETAILED DESCRIPTION

In the following examples, reference will be made to the figures, in which identical features are designated with like numerals.

FIG. 1 shows an exemplary method 1000 for treating biodegradable packaging waste prior to subjecting the biodegradable packaging waste to a biological valorisation process. Method includes subjecting the biodegradable packaging waste to mesophilic aerobic digestion in block 1020 prior to subjecting the biodegradable packaging waste to the biological valorisation process in block 1040. In other words, the method includes a method of digesting biodegradable packaging waste by subjecting the biodegradable packaging waste to mesophilic aerobic digestion, and subsequently subjecting the biodegradable packaging waste to the biological valorisation process.

The biodegradable packaging waste is being pre-treated with mesophilic aerobic digestion before subjecting the treated biodegradable packaging waste with the biological valorisation process. In this way, the biodegradable packaging waste is being converted into a viable feedstock for the biological valorisation process. Biological valorisation process may include enzymatic hydrolysis, fermentation, anaerobic digestion, or composting e.g. industrial composting.

The method may consist of only subjecting the biodegradable packaging waste to mesophilic aerobic digestion prior to subjecting the biodegradable packaging waste to the biological valorisation process without additional steps to digest the biodegradable packaging waste. In other words, the method may consist only the steps of subjecting the biodegradable packaging waste to mesophilic aerobic digestion, and subsequently subjecting the biodegradable packaging waste to the biological valorisation process without additional steps to digest the biodegradable packaging waste. Unlike known methods of deploying multiple stages of digestion, e.g. aerobic digestion and anaerobic digestion or cyclical aerobic digestion and anaerobic digestion stages, the method consist of only a single stage of mesophilic aerobic digestion before a single stage of biological valorisation process and not vice versa.

By subjecting the biodegradable packaging waste to mesophilic aerobic digestion, the waste is broken down into smaller building blocks using aerobic microbial treatment so that the waste can be digested by anaerobic microbes. For example, biodegradable packaging waste may include, but not limited to, biodegradable material like pulp-based material, e.g. paper, moulded fibre, etc. and bioplastics like polylactic acid (PLA), polyhydroxyalkanoates (PHA), Polyhydroxybutyrate (PHB), polybutylene succinate (PBS), thermoplastic starch (TPS). The waste may be broken down to smaller building blocks like lignin, cellulose, lactic acid (LA), glucose and volatile fatty acids (VFA), which can be digested anaerobically. Using this method, a higher yield of biogas is obtained from the anaerobic digestion, i.e. higher amount of biogas per unit of mixed waste.

In addition, by subjecting biodegradable packaging waste to mesophilie aerobic digestion, it is possible to digest the treated biodegradable packaging waste, or the building blocks, using mesophilic anaerobic digestion instead of the conventional anaerobic thermophilic digestion. By using mesophilic anaerobic digestion, it is possible to digest the waste at temperature in the range of about 20° C. to about 45° C. Compared to the temperature of thermophilic anaerobic digestion, which is in the range of 45° C. to about 120° C., the method does not require intensive energy consumption and saves energy when digesting biodegradable packaging waste, aerobically and anaerobically.

Besides energy saving, the method accelerates the degradation of biodegradable packaging waste. Typically, food waste takes about 20-30 days when degraded anaerobically, and biodegradable packaging waste takes a considerably longer period, e.g. 120 days. By pre-treating the biodegradable packaging waste using the method before the biological valorisation process, the time required to degrade the waste is reduced drastically to be about the 20-30 days, i.e. about the same period as the degradation of food waste. Therefore, it makes it viable to allow the biodegradable packaging waste to be degraded together with the food waste and segregation of waste would not be necessary. In this way, it is more commercially sustainable and practical to subject mixed waste to be biodegraded through a biological valorisation process, e.g. anaerobic digestion. On the whole, the method reduces the amount of time required to digest biodegradable packaging waste and hence saves energy, time and cost.

As the mesophilic aerobic digestion uses a mixture of microbial consortium to digest the biodegradable packaging waste, the method does not require addition of catalyst or addictive. Therefore, there is considerable material, energy and cost savings.

Microbial consortium may include a mixture of microbes that has the property of breaking down specific material (e.g., microbes that are able to break down PLA). Hence, the microbial consortium is able to target a specific packaging material. There may be 2 or more microbial consortia, each consisting of microbial strains capable of synthesizing the appropriate enzymes required to hydrolyse specific packaging material in the waste.

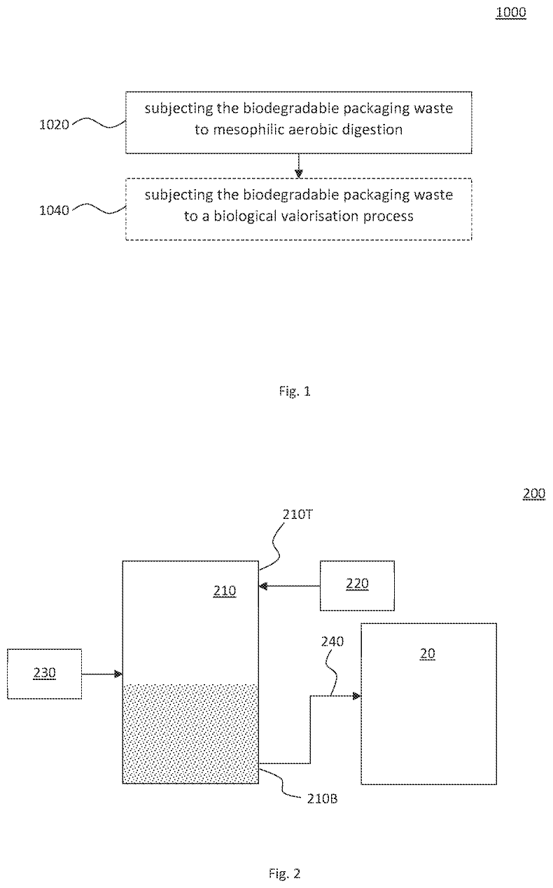

FIG. 2 shows an exemplary embodiment of an apparatus 100 for treating biodegradable packaging waste prior to subjecting the biodegradable packaging waste to the biological valorisation process. Apparatus 100 includes a tank 210 for receiving the biodegradable packaging waste therein, the tank 210 comprises a top portion 210T and a bottom portion 210B below the top portion 210T, an air supply module 220 adapted to supply air into the tank 210, a microbe injector 230 adapted to inject mesophilic microbe into the tank 210, such that the tank 210 is adapted to receive the biodegradable packaging waste, air and mesophilic microbe therein, wherein the biodegradable packaging waste is subjected to mesophilic aerobic digestion prior to conveying the biodegradable packaging waste from the tank 210 to a biological valorisation system 20 to subject the biodegradable packaging waste to the biological valorisation process. In the tank 210, the biodegradable packaging waste is subjected to the method 1000 as shown in FIG. 1. Apparatus 100 may include the biological valorisation system 20.

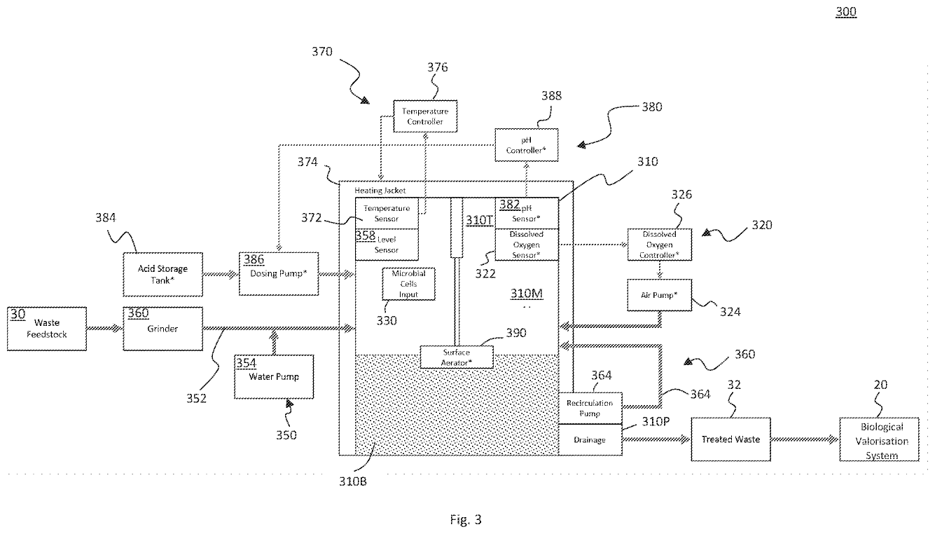

FIG. 3 shows another exemplary embodiment of the apparatus 300. Apparatus 300 may receive the biodegradable packaging waste as a waste feedstock 30 to be conveyed into the tank 310. Waste feedstock 30 may include mixed waste, i.e. food waste and biodegradable packaging waste. Waste feedstock 30 may be fed directly into the tank 310 via a transporting device 350. Transporting device 350 may include a conveyor belt, pressurized conduit, etc. As shown in FIG. 2, the transporting device 350 may include a pump 352 along an inlet conduit 354 connected to the tank 310 and adapted to pump the waste feedstock 30 from a feedstock source into the tank 310. Waste feedstock 30 may be in a sludge form. Apparatus 300 may include a grinder 356 adapted to grind the waste feedstock 30 into smaller particle size before transporting the waste feedstock 30 into the tank 310. Grinder 356 may be connected to transporting device 350 to receive the feedstock and connected to the tank 310 to convey the ground waste feedstock 30 into the tank 310. Apparatus 300 may include a level sensor 358 configure to sense the level of waste in the tank 310. Apparatus 300 may include a waste control module (not shown in FIG. 3) in communication with the transporting device 350 and the level sensor 358 and configured to control the level of waste in the tank 310. Waste control module is configured to receive a signal from the level sensor 358 and transmit a control signal to the transporting device 350. Level sensor 358 upon detecting the waste level is below a pre-determined level, the waste control module is configured to activate the transporting device 350 to convey the feedstock 30 into the tank 310.

Air supply module 320 may include a dissolved oxygen (DO) sensor 322 configured to sense the amount of dissolved oxygen level in the waste in the tank 310, an air pump 352 adapted to pump 352 air or oxygen into the tank 310 and a DO controller in communication with the DO sensor and the pump 352 and configured to control the dissolved oxygen level in the waste. DO controller is configured to receive a signal from the DO sensor and transmit a control signal to the air pump 352. DO sensor upon sensing the level of dissolved oxygen in the waste transmits the signal to the DO controller. If the level of dissolved oxygen is below a pre-determined level, the DO controller in configured to activate the air pump 352 to pump air or oxygen into the tank 310. Air supply module 320 may be configured to supply air into the tank 310 to maintain the mesophilic aerobic digestion of the biodegradable packaging waste at a dissolved oxygen level in the range of about 2 mg/L to about 3 mg/L. Air supply module 320 is configured to continuously monitor the dissolved oxygen level in the waste and supply air when necessary to maintain the level at the pre-determined level. Dissolved oxygen in the waste may be maintained above 2 mg/L to keep the microbes alive. While it is possible to maintain a dissolved oxygen level above 3 mg/L, the dissolved oxygen level is maintained at 3 mg/L or below to lower energy usage. Air supply module 320 may include a backup air pump 352 (not shown in FIG. 3) in communication with the DO controller such that the backup air pump can be activated if the air pump 352 malfunctions.

Microbe injector 230 may include a cultivation tank (not shown in FIG. 3) containing aerobic microbe and an injection pump (not shown in FIG. 3) adapted to inject the aerobic microbe from the cultivation tank into the tank 310. Injection pump may be configured to inject varying dose of aerobic microbes and may be controlled to inject at varying rates as required for the waste.

Apparatus 300 may include a temperature module 370 configured to maintain the mesophilic aerobic digestion of the biodegradable packaging waste at a temperature within a range. Temperature module 370 may include a temperature sensor 372 configured to sense the temperature in the tank 310, a heating element 374 adapted to heat the tank 310 and a temperature controller 376 in communication with the temperature sensor 372 and the heating element 374 and configured to control the temperature in the tank 310. Heating element 374 may be disposed within the tank 310 or around the tank 310, e.g. a heating jacket. Heating element 374 may be disposed above or within the waste. Temperature sensor 372 may be disposed in the waste and configured to sense the temperature of the waste. Tank 310 may be insulated to prevent heat within the tank 310 from escaping into the surrounding. Temperature controller 376 is configured to receive a signal from the temperature sensor 372 and transmit a control signal to the heating element 374 to heat the tank 310. When the temperature sensor 372 senses the temperature in the tank 310, it transmits the signal to the temperature controller 376. If the temperature is below a lower pre-determined temperature, the temperature controller 376 is configured to activate the heating element 374 to heat the tank 310. Similarly, if the temperature is above an upper pre-determined temperature, the temperature controller 376 is configured to inactivate the heating element 374 to stop heating the waste. Temperature module 370 may be configured to maintain the mesophilic aerobic digestion of the biodegradable packaging waste at a temperature in a range of about 20° C. to about 45° C. Preferably, the temperature is maintained at about 20° C. to about 40° C. to reduce the energy usage. Ideally, the temperature is maintained at 20° C. to about 30° C. Temperature module 370 is configured to continuously monitor the temperature in the tank 310 and heat the tank 310 when necessary to maintain the temperature in the tank 310 at the pre-determined temperature. As the digestion is exothermic, the heat released from the digestion of the waste may be sufficient to maintain the temperature of the waste in an insulated tank 390. Hence, it is not required to heat the waste and energy is saved.

Apparatus 300 may include a pH module 380 configured to maintain the mesophilic aerobic digestion of the biodegradable packaging waste at a pH value within a range. pH module 380 may include a pH sensor 382 configured to sense the pH value of the waste, a solution storage tank 384 adapted to store at least one pH adjustment solution, a dosing pump 386 adapted to pump 352 the at least one adjustment solution from the storage tank 384 into the tank 310 and a pH controller 388 in communication with the pH sensor 382 and the dosing pump 386 and configured to control the pH value in the waste. Solution may be an acid or an alkali or both if there is more than one adjustment solution. pH controller is configured to receive a signal from the pH sensor 382 and transmit a control signal to the dosing pump 386 to pump the necessary amount of adjustment solution into the tank 310. When the pH sensor 382 senses the pH value in the waste, it transmits the signal to the pH controller. If the pH value is below a lower pre-determined value, the pH controller is configured to activate the dosing pump 386 to pump the alkali into the tank 310. Similarly, if the pH value is above an upper pre-determined value, the pH controller is configured to activate the dosing pump 386 to pump the acid into the tank 310. pH module 380 may be configured to maintain the mesophilic aerobic digestion of the biodegradable packaging waste at a pH value in the range of about 4 to about 7 to ensure the effective digestion of the waste. Preferably, the pH value of the waste is maintained at about 5 to about 6.5 for optimal rate of degradation. pH module 380 is configured to continuously monitor the pH value of the waste and pump the appropriate amount of adjustment solution when necessary to maintain the pH value of the waste at the pre-determined pH value.

Apparatus 300 may include a surface aerator 390 disposed in the tank 310, such that the surface aerator 390 is adapted to aerate the biodegradable packaging waste and propel the biodegradable packaging waste at the top part of the waste toward the bottom part of the waste. Surface aerator 390 may be disposed at a position in tank 310 that is at the top part of the waste so that the surface aerator 390 is able to contact and propel the top part of the waste toward the bottom portion 310B of the tank 310. Hence, the surface aerator 390 may be at the top portion 310T or an intermediate portion 310M of the tank 310, which is between the top portion 310T and the bottom portion 310B. By aerating the waste, the surface aerator 390 increases the level of dissolved oxygen within the waste for aerobic microbial activities to happen. Aerobic microbes tend to form aggregates, which tend to settle at the bottom of the tank 310. Unlike the traditional way of aeration which introduces air from the bottom of the tank 310 and creates bubbles, thus causing waste with lower density to float to the top of the waste and impede microbial activities, the surface aerator 390 stirs and pushes down the waste that would otherwise float on the surface of the waste in the tank 310 so that the aerobic microbes can be mixed with the waste more homogeneously. Furthermore, the stirring also helps to break up the aggregates for the microbes to be more evenly distributed within the tank 310. This is especially essential for strains of microbes without flagella, which have limited to no mobility and relies on externally induced agitation to reach the waste. Surface aerator 390 may be configured to rotate at different speeds to cater to the rate of mixing required to mix the waste with the adjustment solution, oxygen, and microbes added to the waste. Surface aerator 390 may include an impeller adapted to rotate in a vertical axis and to propel the waste at the top part towards the bottom part.

Apparatus 300 may include a circulating mechanism 360 in fluid communication with the tank 310 and adapted to circulate the biodegradable packaging waste from the bottom part of the waste onto the top part of the waste. Circulating mechanism 360 may include a duct 362 in fluid communication with the tank 310 such that one end of the duct 362 is connected to the bottom portion 310B of the tank 310 and the other end of the duct 362 is connected to the top portion 310T or the intermediate portion 310M of the tank 310 and above the waste. Circulating mechanism 360 may include a circulating pump 364 connected to the duct 362 and adapted to pump the waste from the bottom portion 310B to the top portion 310T or intermediate portion 310M so that the bottom part of the waste is transferred onto the top part of the waste. In addition to the surface aerator 390, the circulation of the waste increases the level of dissolved oxygen within the waste for aerobic microbial activities to happen. As aerobic microbes tend to form aggregates, which tend to settle at the bottom of the tank 310, circulating the waste ensures that the waste is well mixed and any aggregate at the bottom portion 310B of the tank 310 may be transferred to the top part of the waste. Also, the addition of waste to the top part of the waste helps to push the top part of the waste, which may have floating material, down. Circulation pump 364 may be pumping at different rates to vary the rate of circulation of the waste.

Tank 310 may include a drainage port 310P disposed at the bottom portion 310B of the tank 310 and adapted to discharge the waste from the tank 310. The discharged waste may be transported to the biological valorisation system 20. Drainage port 310P may be connectable to a conduit that is connected to the biological valorisation system 20 such that the waste may be conveyed to the biological valorisation system 20 via the drainage port 310P and conduit. The biological valorisation system 20 can be part of an anaerobic digestion plant or industrial composting plant.

Apparatus 300 may include a computing device (not shown in FIG. 3) in communication with the modules, e.g. air supply module 320, temperature module 370, pH module 380. Computing device is configured to receive data and monitoring parameters from the modules. Computing device is configured to record the data and parameters in a database. Computing device may include a programmed module configured to control the modules. Computing device may be configured to control microbe injector 330 to inject the aerobic microbes at programmed time, rate and dosage. Apparatus 300 may include a programmable logic controller (PLC) configured to control the components in the apparatus 300, e.g. surface aerator 390, based on pre-programmed processes. For example, when an adjustment of pH value is required, the dosing pump will inject the adjustment solutions (base or acid) at a controlled rate, with the surface aerator 390 and circulation pump 364 operating at a higher rate concurrently to ensure an even distribution of the adjustment solution, including the microbes, throughout the waste, as quickly as possible. In this way, the waste can be as homogeneous as possible so that the pH sensor is able to measure an accurate pH value of the waste at any point in time. Hence, it is possible to obtain a reading of the pH value of the waste accurately and quickly to enable an appropriate and accurate amount of the adjustment solution to be injected so as to maintain the optimal condition for the aerobic microbes to thrive.

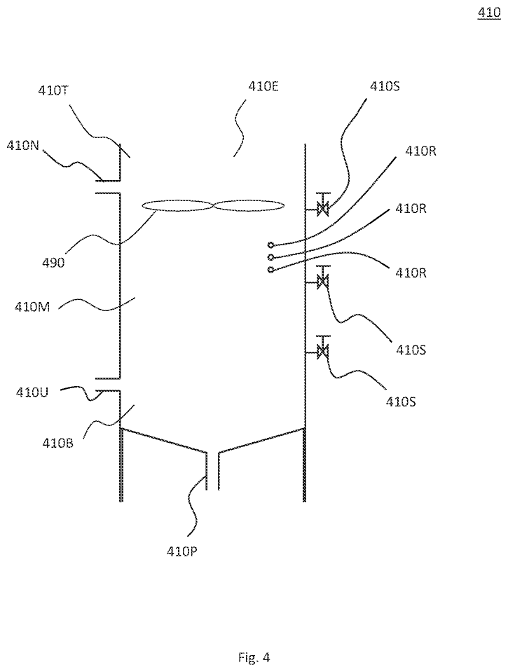

FIG. 4 shows an exemplary embodiment of the tank 410. Tank 410 may include an input port 410N and an output port 410U for channeling the waste into and out of the tank 410. Input port 410N may be disposed at the top portion 410T of the tank 410 above the output port 410U, which may be at the bottom portion 410B of the tank 410. Input port 410N and output port 410U may be connectable to the circulation mechanism (not shown in FIG. 4) to pump the bottom part of the waste from the output port 410U to the top part of the waste via the input port 410N. Tank 410 may include an inlet 410E for receiving the waste therein and the drainage port 410P for discharging the waste. Inlet 410E may be dispose at the top portion 410T or intermediate portion 410M but above the waste level. The base of the tank 410 may be tapered to direct the waste toward the drainage port 410P. Tank 410 may include probe ports 410R for sensors to be inserted into the tank 410. There may be at least three probe ports 410R for the temperature sensor 372, pH sensor 382 and DO sensor. Tank 410 may include at least one sampling port 410S for samples of the waste to be tested or extracted. Sampling port 410S may be disposed at the top portion 410T, bottom portion 410B, and/or the intermediate portion 310M of the tank 410. As mentioned above, the surface aerator 490 and level sensor (not shown in FIG. 4) may be disposed within the tank 410 and a heating element 474 may be disposed in or around the tank 410.

To digest the biodegradable packaging waste, the waste feedstock 30 may be conveyed into the tank 310 via the transporting device 350. Waste feedstock 30 may be broken into smaller particle size by the grinder 356 before entering the tank 310 via the inlet. In the tank 310, the waste is filled to a pre-determined level and the data and parameters of the waste is obtained. If the temperature of the waste is below the pre-determined temperature, the apparatus 300 may activate the heating clement 374 to beat the waste to the pre-determined temperature. If the pH value of the waste is not at the pre-determined value, the apparatus may activate the dosing pump 386 to dose the solution into the waste. If the dissolved oxygen level is below a pre-determined value, the apparatus 300 may activate the air pump 324 to supply air or oxygen into the tank to increase the oxygen level in the tank 310. At the same time, the apparatus 300 may activate the microbe injector 330 to inject the aerobic microbe into the waste. While doing so, the surface aerator 390 is activated to stir and propel the waste and the circulating mechanism 360 is activated to circulate the bottom part of the waste onto the top part of the waste. Apparatus 300 may vary at least the speed of rotation of the surface aerator 390, the pump flowrate of the circulation pump 364, the dosage of the adjustment solution, and the aerobic microbes as required. The waste may be treated in the tank 310 for up to 7 days before conveying it to the biological valorisation system 20. Preferably, the waste may be treated in the tank 310 for 2 to 3 days. Thereafter, the treated waste may be discharged from the tank 310 and conveyed to the biological valorisation system 20.

A skilled person would appreciate that the features described in one example may not be restricted to that example and may be combined with any one of the other examples.

The present invention relates to a method and system for treating biodegradable packaging waste prior to subjecting the biodegradable packaging waste to a biological valorisation process generally as herein described, with reference to and/or illustrated in the accompanying drawings.

1. A method for treating biodegradable packaging waste prior to subjecting the biodegradable packaging waste to a biological valorisation process, the method comprises:
subjecting the biodegradable packaging waste to mesophilic aerobic digestion prior to subjecting the biodegradable packaging waste to the biological valorisation process.

2. The method according to claim 1, wherein the mesophilic aerobic digestion of the biodegradable packaging waste is maintained at a temperature in the range of about 20° C. to about 45° C.

3. The method according to claim 1, wherein the mesophilic aerobic digestion of the biodegradable packaging waste is maintained at a pH value in the range of about 4 to about 7.

4. The method according to claim 1, wherein the mesophilic aerobic digestion of the biodegradable packaging waste is maintained at a dissolve oxygen level in the range of about 2 mg/L to about 3 mg/L.

5. The method according to claim 1, further comprising receiving the biodegradable packaging waste in a tank comprising a top portion and a bottom portion below the top portion, supplying air into the tank, injecting mesophilic microbe into the tank and conveying the biodegradable packaging waste from the tank to a biological valorisation system.

6. The method according to claim 5, further comprising aerating the biodegradable packaging waste and propelling the biodegradable packaging waste from the top portion toward the bottom portion of the tank.

7. The method according to claim 6, further comprising circulating the biodegradable packaging waste from the bottom portion to the top portion of the tank.

8. An apparatus for treating biodegradable packaging waste prior to subjecting the biodegradable packaging waste to a biological valorisation process, the apparatus comprising:
a tank for receiving the biodegradable packaging waste therein, the tank comprises a top portion and a bottom portion below the top portion,
an air supply module adapted to supply air into the tank,
a microbe injector adapted to inject mesophilic microbe into the tank,
wherein the tank is adapted to receive the biodegradable packaging waste, air and mesophilic microbe therein, wherein the biodegradable packaging waste is subjected to mesophilic aerobic digestion prior to conveying the biodegradable packaging waste from the tank to a biological valorisation system to subject the biodegradable packaging waste to the biological valorisation process.

9. The apparatus according to claim 8, further comprising a temperature module configured to maintain the mesophilic aerobic digestion of the biodegradable packaging waste at a temperature in a range of about 20° C. to about 45° C.

10. The apparatus according to claim 8, further comprising a pH module configured to maintain the mesophilic aerobic digestion of the biodegradable packaging waste at a pH value in the range of about 4 to about 7.

11. The apparatus according to claim 8, wherein the air supply module is configured to supply air into the tank to maintain the mesophilic aerobic digestion of the biodegradable packaging waste at a dissolved oxygen level in the range of about 2 mg/L to about 3 mg/L.

12. The apparatus according to claim 8, further comprising a surface aerator disposed in the tank, wherein the surface aerator is adapted to aerate the biodegradable packaging waste and propel the biodegradable packaging waste at the top part of the waste toward the bottom part of the waste.

13. The apparatus according to claim 8, further comprising a circulating mechanism in fluid communication with the tank and adapted to circulate the biodegradable packaging waste from the bottom part of the waste to the top part of the waste.

14. The apparatus according to claim 8, wherein the tank is thermally insulated.

15. A method for digesting biodegradable packaging waste, the method comprising:
subjecting the biodegradable packaging waste to mesophilic aerobic digestion, and
subsequently subjecting the biodegradable packaging waste to a biological valorisation process.
